(19) United States
(12) Patent Application Publication
(10) Pub. No.: US 2005/0125015 A1
(43) Pub. Date: Jun. 9, 2005

(54) Tissue-handling apparatus, system and method

(76) Inventors: Karen M. McNally-Heintzelman, Cambridge, MA (US); Mark T. Duffy, Green Bay, WI (US); Jeffrey N. Bloom, Chicago, IL (US); Douglas L. Heintzelman, Cambridge, MA (US)

Correspondence Address: Intellectual Property Group;Bose McKinney & Evans LLP, 2700 First Indiana Plaza, 135 North Pennsylvania Street, Indianapolis, IN, 46204, US

(21) Appl. No.: 11/003,652

(22) Filed: Dec. 3, 2004

Related U.S. Application Data
(60) Provisional application No. 60/526,855, filed on Dec. 4, 2003.

(51) Int. Cl. A61B 17/08; A61B 17/44

(52) U.S. Cl. 606/151; 606/205

(57) ABSTRACT

A tissue handling apparatus for handling biological tissue is provided. The apparatus includes a handle, at least one clamp, and at least one actuator.

### BACKGROUND

The use of sutures is the mainstay of most forms of modern-day surgery. For example, until very recently, virtually all ophthalmic surgery was only possible with specialized sutures and needles. Strabismus correction, corneal transplantation, vitreoretinal surgery, trauma repair, oculoplastic procedures, glaucoma surgery, and extracapsular cataract removal all require precise suture placement through various fine structures of the eye and adnexa. While microscopes, loupe magnification and specialized illumination techniques have made it easier to perform ophthalmic surgery, suture placement is technically difficult, time-consuming and may be associated with serious complications.

In particular, strabismus surgery requires that a suture be inserted and advanced within the sclera at a depth of approximately half the scleral thickness. This is usually performed with the aid of loupe magnification. Perforation of the sclera during reattachment of an extraocular muscle may be associated with the vision-threatening complications of retinal detachment and endophthalmitis.^{1,2} It has also recently been suggested that contaminated intrascleral sutures may produce endophthalmitis even in the absence of scleral perforation.^{3,4} Suture use in corneal transplantation may be associated with postoperative astigmatism and wound leakage.⁵ Traumatic stellate corneal lacerations can be difficult to close using sutures due to their complex, irregular geometry, while posterior scleral sutures required in the repair of a ruptured globe may be arduous to insert without putting excessive pressure on the eye.

Inherent complications and drawbacks of suture use are numerous. Suture placement can be time-consuming. Sutures must be placed very precisely in order to properly align the tissue. Often the tissue must be manually realigned before each pass of the suture's needle. Imprecise placement of a suture may necessitate its removal and replacement; as a result, delicate ocular tissues may be damaged. Sutures frequently must be removed postoperatively. Not only is this time-consuming, but children often require either restraint, sedation, or additional exposure to general anesthetics. Sutures can also produce allergic reactions and act as a nidus for infection. Finally, sutures pose the risk of needle-stick injury and transmissible infections for operating room personnel.

Currently, the only sutureless ophthalmic procedure is small-incision phacoemulsification of cataractous lenses. Attempts at utilizing bioadhesives in ophthalmic surgery have been limited. At present, the only bioadhesives used in clinical ophthalmic procedures are cyanoacrylates for corneal perforations.^{6,7} Fibrin glue has been used for experimental strabismus surgery in rabbits, but its tensile strength has been disappointing.^{8,9} 2-octyl-cyanoacrylate glue has also been used for rabbit strabismus surgery,¹⁰ as well as attaching rabbit extraocular muscles to porous anophthalmic implants,¹¹ and has demonstrated greater adhesive strength than fibrin glue. However, liquid adhesives may be difficult to precisely position on the sclera and the rapid hardening time after placement requires quick and accurate placement of the muscle. Cyanoacrylate glue has also proven to be of limited efficacy in the repair of leaking filtering blebs.¹²

One major complication limiting acceptance of such adhesives is associated with the difficulty in delivering the surgical adhesive to the repair site. The fluid adhesives tend to run away from the application site, resulting in insufficient quantities of the adhesive being present to provide a strong bond, or potentially worse, tissues adhering together at locations other than that intended.

Improvements to currently available biologic and synthetic adhesives are addressed in a co-pending U.S. Patent application: Non-Light Activated Biological Adhesive Device, System, and Methods of Use Thereof, Ser. No. 10/610,068, filed June 2003 (co-inventors McNally-Heintzelman K M, Heintzelman D L, Bloom J N and Duffy M T). The present application describes two new surgical instruments for handling tissue, particularly muscle tissue, during surgery. These instruments are intended for use in ophthalmic surgeries as well as other types of internal or external surgery. At least one embodiment is designed to facilitate the use of an adhesive composite such as is described in the above-mentioned U.S. patent application, and other surgical adhesives and adhesive-enhanced repair techniques.

While the present invention has been described with respect to particular applications in ophthalmic procedures, it is understood that it is capable of broader application, for example, in other types of surgical procedures.

### SUMMARY

One aspect of the present invention relates to an apparatus, system and method for facilitating the handling of tissue, particularly muscle tissue, during surgical procedures. Strabismus surgery, for example, commonly entails recessions of eye muscles if weakening of muscles is required, and resections of eye muscles when strengthening of eye muscles is required. Recession of an eye muscle requires disinsertion of one of the six muscles attached to the eye, and reattachment of the muscle further back on the eye, thereby causing weakening. Resection of an eye muscle requires disinsertion of the muscle from the eye, excising a portion of the distal end of the muscle, and subsequent reattachment to the eye, thereby resulting in a stronger muscle.

Prior to disinsertion, a suture is typically pre-placed in the muscle, by threading it through the tissue, in order to secure the muscle after it is disinserted from the sclera and prevent it from retracting back into the orbit. Thus, even if a suitable adhesive is available, the surgery is not truly sutureless. Suture location within the muscle may vary between surgeons, or even for the same surgeon, producing variable surgical outcomes. The needle of the suture may also damage the muscle as it is passed through it and poses a risk of scleral perforation during this pre-placement within the muscle tissue. In addition, improperly locating the suture within the muscle may cause the suture to pull through the muscle and allow the muscle to retract back into the orbit, producing the serious complication of a “lost” muscle. The present invention provides a sutureless method for precisely holding the muscle during the disinsertion and reinsertion process.

Another aspect of the present invention relates to a clamp that is configured to keep a muscle spread to its full tendon width, allowing the tendon edge to be precisely placed against the adhesive material. In contrast, with the use of a suture to hold the muscle, it is difficult to maintain a perfectly spread muscle, with narrowing of the muscle width typically occurring at the time it is reattached to the sclera. This bunching-up of tissue can lead to incorrect placement of the muscle on the surgical adhesive.

Yet another aspect of the present invention relates to the atraumatic use of non-slip teeth or tooth-like structures to hold the muscle at strategic points, thereby providing a non-crushing grip of the tendon or muscle.

Yet another aspect of the present invention relates to a method of application that provides for an accelerated surgical procedure. According to one embodiment of the method, a clamp is placed on the muscle immediately prior to its disinsertion, and remains in place during the entire procedure. The clamp is configured to facilitate release and reattachment of the muscle without re-gripping or damaging the tissue.

Another aspect of the invention involves an alternative design of a clamp for use in connection with an apparatus for handling muscle tissue. The alternative clamp is configured to allow more user control over the disinserted muscle. In one embodiment, the clamp includes a “dual-action” feature. In another embodiment, the alternative clamp is configured to reduce the amount of muscle manipulation needed to secure the muscle. In a further embodiment, the clamp has an adjustable width. In still another embodiment, the clamp is configured to reduce the amount of necessary contact with the muscle.

Another alternative form of the apparatus of the present invention is configured to facilitate application of an adhesive composite to tissue to achieve a desired placement of the composite on the tissue. For example, in strabismus surgery, it may be desired to position the composite as a bridge to ophthalmic tissues such as the sclera. This configuration allows for one-step attachment of the muscle to the sclera (versus the known two-step process of first placing the adhesive on the sclera and then placing the muscle on top of the adhesive).

### BRIEF DESCRIPTION OF THE DRAWINGS

FIG. 1 is a side view of a first embodiment of an apparatus for handling tissue.

FIG. 2 is a top view of a second alternative embodiment of an apparatus for handling tissue.

### DETAILED DESCRIPTION

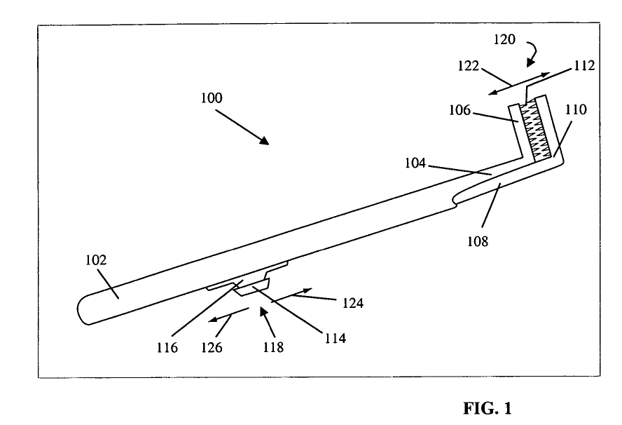

FIG. 1 shows a tissue handling apparatus 100 designed to facilitate the handling of muscle tissue in particular. The illustrated embodiment is configured to hold muscle in an extended (unretracted) position. Preferably, in ophthalmic surgeries, the muscle is spread to its full tendon width, and the illustrated embodiment of tissue handling apparatus 100 is designed to be capable of holding muscle in such a fully-extended position.

Tissue handling apparatus 100 includes a handle such as an elongated member 102. The handle 102 has a substantially L-shaped end portion having a clamp end 104 and a first clamp portion 106. As shown, elongated member 102, clamp end 104, and first clamp portion 106 are of substantially one-piece (e.g., molded) construction. It is understood, however, that clamp end 104 may be coupled to elongated member 102 and first clamp portion 106 by any suitable means known in the art (e.g., solder, adhesive, fasteners, etc.). First clamp portion 106 and second clamp portion 110 form clamp 120.

Also coupled to the handle 102 is an actuator 118 for operating the clamp 120. As illustrated, coupled to the elongated member 102 are slide mechanism 118 and sliding member 108. Slide mechanism 118 operates to move sliding member 108 back and forth in the directions of arrow 122. Sliding member 108 slides back and forth within a hollowed portion, slot or track (not shown) of elongated member 102.

Slide mechanism 118 includes a slide 116 and a slide actuator 114. When not actuated, slide mechanism 118, and consequently sliding member 108, are in a locked position. When pressure is applied to slide actuator 114, slide mechanism unlocks and slide 116 becomes movable in the directions of arrows 124 and 126.

For example, pressure may be applied to slide actuator 114 via a thumb or index finger of a medical professional. Pressing downwardly on slide actuator 114 unlocks slide mechanism 118. Releasing pressure from slide actuator 114 causes slide mechanism 118 to be disabled or locked.

Slide 116 is mechanically coupled to sliding member 108 by suitable means known in the art, such that slide 116 slides along the slot or track discussed above. Movement of slide 116 in the direction of arrow 124 causes sliding member 108 to move in the direction away from elongated member 102, widening the space between clamp portions 106 and 110. Movement of slide 116 in the direction of arrow 126 causes sliding member 108 to move toward elongated member 102, narrowing the gap between clamp portions 106 and 110.

It will be appreciated by those skilled in the art that other suitable mechanical or electrical means for adjusting clamp 120 may be used equally as well as slide mechanism 118.

Each of clamp portions 106, 110 includes at least one tissue grip portion such as protrusion 112 extending outwardly from its inner edge toward the opposite blade. Protrusions 112 are configured to grip the tissue without slipping and without crushing or otherwise damaging the tissue. In the illustrated embodiment, protrusions 112 are substantially tooth-like in shape. It is understood that protrusions 112 may taper to a point and thus be substantially cone-like or pyramid-like in shape. Alternatively, protrusions 112 may be untapered and have a flat, rectangular or cube-like shape.

During a surgical procedure, the above-described apparatus 100 is suitable for use as follows. After the targeted tissue is isolated, clamp 120 is placed on the tissue. The position of second clamp portion 110 is adjusted using slide mechanism 118 to ensure that protrusions 112 engage the tissue at the desired points. Once clamp 120 is in place, slide mechanism 118 is disabled or locked as described above so that clamp 120 remains in the desired position during the duration of the surgery.

In the case of muscle tissue, the positioning of clamp 120 keeps the muscle spread to its full tendon width. Consequently, the surgical procedure, including release and reattachment of the muscle, can be performed, and an adhesive can be applied to the affected area, while clamp 120 remains in place as originally positioned.

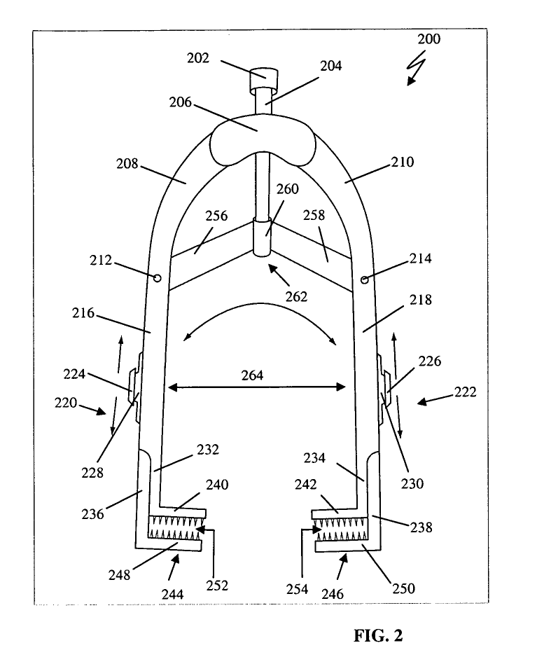

In FIG. 2, an alternative “dual-action” embodiment 200 of a tissue-handling apparatus in accordance with the present invention is shown. Tissue-handling apparatus 200 includes first and second handle portions or clamp arms 216, 218 and first and second clamps 244,246 operable via first and second slide mechanisms 220, 222. As illustrated, the clamp arms 216, 218 are substantially parallel to one another.

Each clamp arm 216, 218 includes a respective curved portion 208, 210. Curved portions 208, 210 are coupled together via coupler or end portion 206.

A width adjuster 262 is also provided. Width adjuster 262 includes first and second cross members 256, 258, which are coupled to each other and post 204 via coupler 260. Each cross member 256, 258 is coupled to the respective clamp arm 216, 218 via a fastener 212, 214. Fasteners 212, 214 may be screws, nuts, or other suitable fasteners known in the art. Alternatively, a solder or adhesive may be used to couple first and second cross members 256, 258 to clamp arms 216, 218. Cap 202 is provided on the end of post 204 opposite coupler 260.

As shown, first and second cross members 256, 258 and coupler 260 are of substantially one-piece construction, coupler 260 is a hollow cylinder, and one end of post 204 fits within the hollow portion of coupler 260. However, it is understood that any other suitable electrical or mechanical means for adjusting the width 264 between clamp arms 216, 218 may be used equally as well.

Each clamp 244, 246 includes a first clamp portion 240, 242 coupled respectively to a clamp end 232, 234, second clamp portions 248, 250, and protrusions 252, 254. Second clamp portions 248, 250 are coupled to or part of sliding members 236, 238.

Sliding members 236, 238 are operable to adjust the positioning of clamps 244, 246 via sliding mechanisms 220, 222 as discussed above. Sliding mechanisms 220, 222 include slide actuators 224, 226 and slides 228, 230, which operate as described above.

During a surgical procedure, tissue-handling apparatus 200 is suitable for use as follows. Either or both of clamps 244, 246 are used as described above to secure the tissue in the desired position. Forceps or other suitable means are used to place a biological scaffolding or adhesive composite on the tissue in a desired location, for example, to join two pieces of tissue (i.e., edges of a wound) or to close a wound.

For example, in ophthalmic surgery, one or both of clamps 244, 246 are used to position and hold the muscle in the desired location with respect to the sclera. The choice to use one or two clamps may depend on surgeon preference and/or style of muscle-adhesive-sclera apposition. A biological scaffolding or adhesive composite is then placed directly over the area where the muscle and sclera are to be rejoined (i.e., so the composite acts as a “bridge”), thereby allowing the respective edges of the muscle and sclera to be properly aligned and facilitating healing.

The various embodiments of tissue handling apparatus 100, 200 are made of surgical steel or other suitable material known in the art. It is understood that certain portions of tissue handling apparatus 100, 200 may be coated or enclosed in a synthetic material such as a foam, rubber or other suitable high-friction material to provide for easier maintenance and handling by the medical professional.

Although the present invention has been described in detail with reference to certain exemplary embodiments, it is understood that variations and modifications exist and are within the scope and spirit of the present invention.

### REFERENCES

All of the following references, whether or not specifically cited elsewhere in this disclosure, are incorporated herein by this reference.
1. Noel L P, Bloom J N, Clarke W N, Bawazer A. Retinal perforation in strabismus surgery. J Pediatr Ophthalmol Strabismus. 1997;34:115-117.
2. Awad A H, Mullaney P B, Al-Hazmi A, Al-Turkmani S, Wheeler D, Al-Assaf M, et al. Recognized globe perforation during strabismus surgery: incidence, risk factors, and sequelae. JAAPOS. 2000;4:150-153.
3. Recchia F M, Baumal C R, Sivalingam A, Keiner R, Duker J S, Vrabec T R. Endophthalmitis after pediatric strabismus surgery. Ophthalmol. 2000;118:939-944.
4. Rosenbaum AL. Endophthalmitis after strabismus surgery. Arch Ophthalmol. 2000;118:982-983.
5. Whitson W E, Weisenthal R W, Krachmer J H. Penetrating keratoplasty and keratoprosthesis; Tasman W, Jaeger E A (ed.); Duane's Clinical Ophthalmology, Philadelphia, Lippincott Williams and Wilkins; 2001, vol. 6, chap. 26, pp 1-28.
6. Vote B J, Elder M J. Cyanoacrylate glue for corneal perforations: a description of a surgical technique and a review of the literature. Clin Experiment Ophthalmol. 2000;28:437-442.
7. Taravella M J, Chang CD. 2-Octyl cyanoacrylate medical adhesive in treatment of a corneal perforation. Cornea. 2001;20:220-221.
8. Spierer A, Barequet I, Rosner M, Solomon A S, Martinowitz U. Reattachment of extraocular muscles using fibrin glue in a rabbit model. Invest Ophthalmol Vis Sci. 1997;38:543-546.
9. Erbil H, Sinav S, Sullu Y, Kandemir B. An experimental study on the use of fibrin sealants in strabismus surgery. Turk J. Pediatr.1991;33:111-116.
10. Ricci B, Ricci F, Bianchi P E. Octyl 2-cyanoacrylate in sutureless surgery of extraocular muscles: an experimental study on the rabbit model. Graefe's Arch Clin Exp Ophthalmol. 2000;238:454-458.
11. Gupta B K, Edward D, Duffy M T. 2-Octyl cyanoacrylate tissue adhesive and muscle attachment to porous anophthalmic orbital implants. Ophthal Plast Reconstr Surg. 2001;17:264-269.
12. Burnstein A L, WuDunn D, Knotts S L, Catoira Y, Cantor L B. Conjunctival advancement versus nonincisional treatment for late-onset glaucoma filtering bleb leaks. Ophthalmology. 2002;109:71-75.

1. An apparatus for handling biological tissue, comprising:
a handle,
a clamp coupled to the handle, and
an actuator coupled to the clamp,
wherein the clamp includes a first clamp member, a second clamp member spaced apart from the first clamp member, a first grip portion coupled to the first clamp member, and a second grip portion coupled to the second clamp member, the actuator being operable to adjust the space between the first and second clamp members.

2. The apparatus of claim 1, wherein the actuator is a slide mechanism.

3. The apparatus of claim 2, wherein the first and second grip portions each include a plurality of protrusions.

4. The apparatus of claim 3, wherein the protrusions are substantially tooth-shaped.

5. The apparatus of claim 1, wherein the handle includes a first handle portion and a second handle portion coupled together at one end by a coupler.

6. The apparatus of claim 5, wherein the clamp is coupled to the first handle portion, and further comprising a second clamp coupled to the second handle portion.

7. The apparatus of claim 5, wherein the first handle portion and the second handle portion are substantially parallel to one another.

8. The apparatus of claim 6, further comprising a second actuator coupled to the second clamp.

9. The apparatus of claim 7, further comprising a width adjuster coupled to the first handle portion and the second handle portion.

10. A method for holding biological tissue using the apparatus of claim 1 during a surgical procedure, comprising the steps of:
positioning biological tissue in a desired location using the clamp,
performing the surgical procedure, and
removing the clamp.

11. The method of claim 10, wherein the biological tissue is muscle tissue and the clamp is used to extend the muscle tissue and hold the extended muscle tissue in place.

12. The method of claim 11, wherein the surgical procedure is strabismus surgery.

13. The method of claim 10, further comprising the step of operating the actuator to position the clamp with respect to the biological tissue.

14. The method of claim 10, wherein the apparatus includes a second actuator, further comprising the step of operating the actuators to position the clamp with respect to biological tissue.

15. A system for closing a wound, comprising:
a tissue handling apparatus including a handle and first and second spaced-apart clamps coupled to the handle, and
a biological adhesive composite.

16. A method for closing a wound using the system of claim 15, comprising the steps of:
positioning biological tissue in a desired location using the first and second clamps,
performing a medical procedure,
applying the adhesive composite to at least a portion of the biological tissue, and
releasing the first and second clamps.
